(19) United States
(12) Patent Application Publication
(10) Pub. No.: US 2020/0384004 A1
(43) Pub. Date: Dec. 10, 2020

(54) METHODS AND COMPOSITIONS FOR TREATING GLAUCOMA

(71) Applicant: Elysium Health, Inc., New York, NY (US)

(72) Inventors: Eric Marcotulli, New York, NY (US); Dan Alminana, New York, NY (US); Mark Morris, New York, NY (US); Ryan Dellinger, Azusa, CA (US)

(21) Appl. No.: 16/768,267

(22) PCT Filed: Nov. 30, 2018
(86) PCT No.: PCT/US18/63204

§ 371 Date: May 29, 2020

Related U.S. Application Data
(60) Provisional application No. 62/593,608, filed on Dec. 1, 2017.

(51) Int. Cl. A61K 31/706; A61P 27/02; A61K 31/09

(52) U.S. Cl. CPC A61K 31/706; A61K 45/06; A61K 31/09; A61P 27/02

(57) ABSTRACT

Provided herein are methods and compositions related to treating and/or preventing glaucoma in a subject by administering to the subject (e.g., orally administering to the subject) a composition comprising nicotinamide riboside and/or pterostilbene.

### CROSS-REFERENCE TO RELATED APPLICATIONS

This application claims the benefit of U.S. Provisional Application No. 62/593,608, filed on Dec. 1, 2017; hereby incorporated by reference in its entirety.

### BACKGROUND

Glaucoma is an eye disorder characterized by increased intraocular pressure, excavation of the optic nerve and gradual loss of the visual field. An abnormally high intraocular pressure is commonly known to be detrimental to the eye, and there are clear indications that, in glaucoma patients, this probably is the most important factor causing degenerative changes in the retina. Unless treated successfully, glaucoma will lead to eventually lead to blindness. There is currently no cure for glaucoma. Thus, novel and innovative therapies are currently needed to treat glaucoma.

### SUMMARY

In some aspects, the methods and compositions disclosed herein relate to treating and/or preventing diseases and disorders characterized by the progressive dysfunction and loss of retinal ganglion cells, eventually leading to vision loss. Such disorders include glaucoma. Also provided herein are methods and compositions related to slowing the progression of glaucoma and/or for treating and/or preventing a symptom or symptoms of glaucoma in a subject by administering to the subject (e.g., orally administering to the subject) a composition comprising a compound of Formula I or Formula II (e.g., nicotinamide riboside), and/or a compound of Formula III (e.g., pterostilbene). In some aspects, provided herein are methods and compositions related to reducing progressive dysfunction or loss of retinal ganglion cells and/or for treating and/or reducing intraocular eye pressure or inflammation in a subject in need thereof, by administering to the subject (e.g., orally administering to the subject) a composition comprising a compound of Formula I or Formula II (e.g., nicotinamide riboside), and/or a compound of Formula III (e.g., pterostilbene). The glaucoma may be open angle glaucoma, angle closure glaucoma, normal tension glaucoma, secondary glaucoma, pigmentary glaucoma, pseudoexfoliative glaucoma, traumatic glaucoma, neovascular glaucoma, irido corneal endothelial syndrome (ICE), or uveitic glaucoma.

A symptom of glaucoma may include, but is not limited to, hazy or blurred vision, appearance of rainbow-colored circles around bright lights, severe eye and head pain, nausea or vomiting, or sudden sight loss. In some embodiments, a composition comprising a compound of Formula I or Formula II (e.g., nicotinamide riboside), and/or a compound of Formula III (e.g., pterostilbene) may be administered to the subject conjointly with a second therapeutic for the treatment of galucoma. The second therapeutic may be a beta-blocker, a alpha-adrenergic agonist, a carbonic anhydrase inhibitor, a parasympathomimetic, epinephrine, or a hyperosmotic agent.

In certain embodiments of the compositions and methods provided herein, the composition comprises a compound of Formula I or Formula II (e.g., nicotinamide riboside) (e.g., at least 100 mg, at least 125 mg, at least 150 mg, at least 175 mg, at least 200 mg, at least 225 mg, at least 250 mg, at least 275 mg, at least 300 mg, at least 325 mg, at least 350 mg, at least 375 mg, at least 400 mg, at least 425 mg, at least 450 mg, at least 475 mg, at least 500 mg, at least 525 mg, at least 550 mg, at least 575 mg or at least 600 mg of a compound of Formula I or Formula II (e.g., nicotinamide riboside)). In some embodiments, the composition comprises a compound of Formula III (e.g., pterostilbene) (e.g., at least 15 mg, at least 20 mg, at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, at least 100 mg, at least 125 mg or at least 150 mg of a compound of Formula III (e.g., pterostilbene)). In certain embodiments, the composition comprises both a compound of Formula I or Formula II (e.g., nicotinamide riboside) (e.g., at least 100 mg, at least 125 mg, at least 150 mg, at least 175 mg, at least 200 mg, at least 225 mg, at least 250 mg, at least 275 mg, at least 300 mg, at least 325 mg, at least 350 mg, at least 375 mg, at least 400 mg, at least 425 mg, at least 450 mg, at least 475 mg, at least 500 mg, at least 525 mg, at least 550 mg, at least 575 mg or at least 600 mg of a compound of Formula I or Formula II (e.g., nicotinamide riboside)) and a compound of Formula III (e.g., pterostilbene) (e.g., at least 15 mg, at least 20 mg, at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, at least 100 mg, at least 125 mg or at least 150 mg of a compound of Formula III (e.g., pterostilbene)).

In certain embodiments, the method comprises administering a plurality of doses of the composition. In some embodiments, at least 7 doses of the composition are administered. In some embodiments, at least 30 doses of the composition are administered. In some embodiments, at least 60 or more doses of the composition are administered. In some embodiments, each dose comprises at least 100 mg, at least 125 mg, at least 150 mg, at least 175 mg, at least 200 mg, at least 225 mg, at least 250 mg, at least 275 mg, at least 300 mg, at least 325 mg, at least 350 mg, at least 375 mg, at least 400 mg, at least 425 mg, at least 450 mg, at least 475 mg, at least 500 mg, at least 525 mg, at least 550 mg, at least 575 mg or at least 600 mg of a compound of Formula I or Formula II (e.g., nicotinamide riboside). In some embodiments, each dose comprises at least 15 mg, at least 20 mg, at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, at least 100 mg, at least 125 mg or at least 150 mg of a compound of Formula III (e.g., pterostilbene). In certain embodiments, each dose comprises at least 100 mg, at least 125 mg, at least 150 mg, at least 175 mg, at least 200 mg, at least 225 mg, at least 250 mg, at least 275 mg, at least 300 mg, at least 325 mg, at least 350 mg, at least 375 mg, at least 400 mg, at least 425 mg, at least 450 mg, at least 475 mg, at least 500 mg, at least 525 mg, at least 550 mg, at least 575 mg or at least 600 mg of a compound of Formula I or Formula II (e.g., nicotinamide riboside) at least 15 mg, at least 20 mg, at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, at least 100 mg, at least 125 mg or at least 150 mg of a compound of formula III (e.g., pterostilbene).

In certain embodiments, a dose of the composition is administered at regular intervals over a period of time. In some embodiments, a dose of the composition is administered at least once a week. In some embodiments, a dose of the composition is administered at least twice a week. In certain embodiments, a dose of the composition is administered at least three times a week. In some embodiments, a dose of the composition is administered at least once a day. In some embodiments, a dose of the composition is administered at least twice a day. In some embodiments, doses of the composition are administered for at least 1 week, for at least 2 weeks, for at least 3 weeks, for at least 4 weeks, for at least 1 month, for at least 2 months, for at least 3 months, for at least 4 months, for at least 5 months, for at least 6 months or for at least 1 year.

In certain embodiments, the composition is formulated for oral delivery. In some embodiments, the composition is formulated as a pill, a tablet, or a capsule. In some embodiments, the composition is administered orally. In certain embodiments, the composition is self-administered.

### DETAILED DESCRIPTION

### General

In some aspects, the methods and compositions disclosed herein relate to treating and/or preventing diseases and disorders characterized by vision loss, such as glaucoma. Also provided herein are methods and compositions related to slowing the progression of glaucoma and/or for treating and/or preventing a symptom or symptoms of glaucoma in a subject by administering to the subject (e.g., orally administering to the subject) a composition comprising a compound of Formula I or Formula II (e.g., nicotinamide riboside), and/or a compound of Formula III (e.g., pterostilbene). In some aspects, provided herein are methods and compositions related to reducing progressive dysfunction and/or loss of retinal ganglion cells in a subject in need thereof (e.g., a subject with glaucoma), by administering to the subject (e.g., orally administering to the subject) a composition comprising a compound of Formula I or Formula II (e.g., nicotinamide riboside), and/or a compound of Formula III (e.g., pterostilbene). In some aspects, provided herein are methods and compositions related to treating and/or reducing intraocular eye pressure and/or inflammation in a subject in need thereof (e.g., a subject with glaucoma), by administering to the subject (e.g., orally administering to the subject) a composition comprising a compound of Formula I or Formula II (e.g., nicotinamide riboside), and/or a compound of Formula III (e.g., pterostilbene).

### Definitions

For convenience, certain terms employed in the specification, examples, and appended claims are collected here.

The articles “a” and “an” are used herein to refer to one or to more than one (i.e., to at least one) of the grammatical object of the article. By way of example, “an element” means one element or more than one element.

As used herein, the term “administering” means providing a pharmaceutical agent or composition to a subject, and includes, but is not limited to, administering by a medical professional and self-administering. Administration of a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

The phrase “pharmaceutically-acceptable carrier” as used herein means a pharmaceutically-acceptable material, composition or vehicle, such as a liquid or solid filler, diluent, excipient, or solvent encapsulating material.

As used herein, the term “subject” means a human or non-human animal selected for treatment or therapy.

The phrases “therapeutically-effective amount” and “effective amount” as used herein means the amount of an agent which is effective for producing the desired therapeutic effect in at least a sub-population of cells in a subject at a reasonable benefit/risk ratio applicable to any medical treatment.

“Treating” a disease in a subject or “treating” a subject having a disease refers to subjecting the subject to a pharmaceutical treatment, e.g., the administration of a drug, such that at least one symptom of the disease is decreased or prevented from worsening.

As used herein, a therapeutic that “prevents” a disorder or condition refers to a compound that, when administered to a statistical sample prior to the onset of the disorder or condition, reduces the occurrence of the disorder or condition in the treated sample relative to an untreated control sample, or delays the onset or reduces the severity of one or more symptoms of the disorder or condition relative to the untreated control sample.

### Compositions

Provided herein are pharmaceutical compositions comprising a compound of Formula I or Formula II (e.g., nicotinamide riboside) and/or a compound of Formula III (e.g., pterostilbene).

Nicotinamide riboside is a pyridine-nucleoside form of niacin (i.e., vitamin B₃) that serves as a precursor to nicotinamide adenine dinucleotide (NAD⁺). As used herein, “nicotinamide riboside” also includes nicotinamide riboside salts, such as nicotinamide riboside chloride. The chemical structure of nicotinamide riboside is provided below:

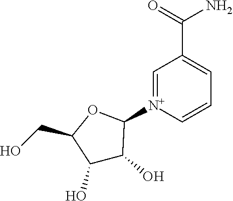 [SMILES: NC(=O)c1ccc[n+]([C@@H]2O[C@H](CO)[C@@H](O)[C@H]2O)c1]

In some embodiments, provided herein are pharmaceutical compositions comprising a compound represented by Formula (I) or a pharmaceutically acceptable salt thereof:

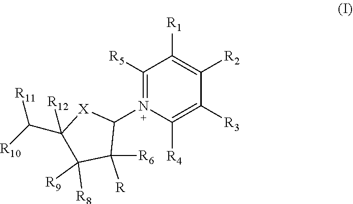

wherein, independently for each occurrence:

R₁, R₂, and R₃ are selected from hydrogen, halogen, —CN, —NO₂, —OR₁₄, —N(R₁₄)ₘ, —R₁₃, substituted or unsubstituted (C₁-C₆)alkyl, cycloalkyl, heterocycloalkyl, aryl, heteroaryl, aralkyl, and heteroaralkyl;

R₄ and R₅ are selected from hydrogen, halogen, —CN, —NO₂, —OR₁₄, —N(R₁₄)ₘ, substituted or unsubstituted (C₁-C₆)alkyl, cycloalkyl, heterocycloalkyl, aryl, heteroaryl, aralkyl, and heteroaralkyl;

R₆, R₈, R₁₁, and R₁₂ are selected from hydrogen, (C₁-C₆)alkyl, —((C₁-C₆)alkylene)N(R₁₄)ₘ, —C(O)((C₁-C₆)alkylene)N(R₁₄)ₘ, cycloalkyl, heterocycloalkyl, aryl, heteroaryl, aralkyl, heteroaralkyl, —OR₁₄, and —N(R₁₄)ₘ;

R₇, R₉, and R₁₀ are selected from —((C₁-C₆)alkylene)N(R₁₄)ₘ, —OR₁₄, and —N(R₁₄)ₘ;

R₁₃ is selected from —OR₁₄, —N(R₁₄)ₘ, —C(O)(R₁₄), —C(O)(OR₁₄), —C(O)N(R₁₄)ₘ, —S(O)₂(OR₁₄), —S(O)OR₁₄, and —S(O)₂N(R₁₄)ₘ;

R₁₄ is selected from hydrogen, (C₁-C₆)alkyl, cycloalkyl, heterocycloalkyl, aryl, heteroaryl, aralkyl, and heteroaralkyl; and

X is O, S, or N(R₁₄);

m is 2 or 3;

provided that at least one of R₁, R₂, and R₃ is R₁₃.

In some embodiments, R₁ is R₁₃. In some embodiments, R₂ is R₁₃. In some embodiments, R₃ is R₁₃.

In some embodiments, R₁₃ is selected from —OR₁₄, —N(R₁₄)ₘ, —C(O)(R₁₄), —C(O)(OR₁₄), and —C(O)N(R₁₄)ₘ. In some embodiments, R₁₃ is selected from —C(O)(R₁₄), —C(O)(OR₁₄), and —C(O)N(R₁₄)ₘ. In some embodiments, R₁₃ is —C(O)N(R₁₄)ₘ.

In some embodiments, R₇, R₉, and R₁₀ are each independently —OR₁₄ or —N(R₁₄)ₘ. In some embodiments, R₇, R₉, and R₁₀ are —OR₁₄.

In some embodiments, the compound of formula (I) is represented by Formula (II) or a pharmaceutically acceptable salt thereof:

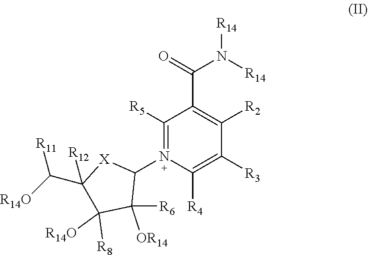

wherein, independently for each occurrence:

R₂ and R₃ are selected from hydrogen, halogen, —CN, —NO₂, —OR₁₄, —N(R₁₄)ₘ, —R₁₃, substituted or unsubstituted (C₁-C₆)alkyl, cycloalkyl, heterocycloalkyl, aryl, heteroaryl, aralkyl, and heteroaralkyl;

R₄ and R₅ are selected from hydrogen, halogen, —CN, —NO₂, —OR₁₄, —N(R₁₄)ₘ, substituted or unsubstituted (C₁-C₆)alkyl, cycloalkyl, heterocycloalkyl, aryl, heteroaryl, aralkyl, and heteroaralkyl;

R₆, R₈, R₁₁, and R₁₂ are selected from hydrogen, —OR₁₄, —N(R₁₄)ₘ, substituted or unsubstituted (C₁-C₆)alkyl, —((C₁-C₆)alkylene)N(R₁₄)ₘ, —C(O)((C₁-C₆)alkylene)N(R₁₄)ₘ, cycloalkyl, heterocycloalkyl, aryl, heteroaryl, aralkyl, and heteroaralkyl;

R₁₃ is selected from —OR₁₄, —N(R₁₄)ₘ, —C(O)(R₁₄), —C(O)(OR₁₄), —C(O)N(R₁₄)ₘ, —S(O)₂(OR₁₄), —S(O)OR₁₄, and —S(O)₂N(R₁₄)ₘ;

R₁₄ is selected from hydrogen, (C₁-C₆)alkyl, cycloalkyl, heterocycloalkyl, aryl, heteroaryl, aralkyl, and heteroaralkyl; and

m is 2 or 3.

In some embodiments of the compounds of formula (I) or (II), R₁, R₂, and R₃ are each independently, if present, selected from hydrogen, halogen, —CN, —NO₂, —OR₁₄, —N(R₁₄)ₘ, —R₁₃, and substituted or unsubstituted (C₁-C₆)alkyl. In some embodiments, R₁, R₂, and R₃ are each independently, if present, selected from hydrogen, —OR₁₄, —N(R₁₄)ₘ, and unsubstituted (C₁-C₆)alkyl. In some embodiments, R₁, R₂, and R₃ are each independently, if present, selected from substituted or unsubstituted (C₁-C₆)alkyl, cycloalkyl, heterocycloalkyl, aryl, heteroaryl, aralkyl, and heteroaralkyl. In some embodiments, R₁, R₂, and R₃ are each independently, if present, hydrogen.

In some embodiments of the compounds of formula (I) or (II), R₄ and R₅ are each independently selected from hydrogen, halogen, —CN, —NO₂, —OR₁₄, —N(R₁₄)ₘ, and substituted or unsubstituted (C₁-C₆)alkyl. In some embodiments, R₄ and R₅ are each independently selected from hydrogen, —OR₁₄, —N(R₁₄)ₘ, and unsubstituted (C₁-C₆)alkyl. In some embodiments, R₄ and R₅ are each independently selected from substituted or unsubstituted (C₁-C₆)alkyl, cycloalkyl, heterocycloalkyl, aryl, heteroaryl, aralkyl, and heteroaralkyl. In some embodiments, R₄ and R₅ are each hydrogen.

In some embodiments of the compounds of formula (I) or (II), R₆, R₈, R₁₁, and R₁₂ are selected from hydrogen, —OR₁₄, —N(R₁₄)ₘ, unsubstituted (C₁-C₆)alkyl, —((C₁-C₆)alkylene)N(R₁₄)ₘ, —C(O)((C₁-C₆)alkylene)N(R₁₄)ₘ, cycloalkyl, heterocycloalkyl, aryl, heteroaryl, aralkyl, and heteroaralkyl. In some embodiments, R₆, R₈, R₁₁, and R₁₂ are each independently selected from hydrogen, —OR₁₄, —N(R₁₄)ₘ, unsubstituted (C₁-C₆)alkyl, —((C₁-C₆)alkylene)N(R₁₄)ₘ, and —C(O)((C₁-C₆)alkylene)N(R₁₄)ₘ. In some embodiments, R₆, R₈, R₁₁, and R₁₂ are each independently selected from hydrogen, —OR₁₄, and —N(R₁₄)ₘ. In some embodiments, R₆, R₈, R₁₁, and R₁₂ are each independently selected from unsubstituted (C₁-C₆)alkyl, cycloalkyl, heterocycloalkyl, aryl, heteroaryl, aralkyl, and heteroaralkyl. In some embodiments, R₆, R₈, R₁₁, and R₁₂ are each hydrogen.

In some embodiments, R₇, R₉, and R₁₀ are each independently —OR₁₄ or —N(R₁₄)ₘ. In some embodiments, R₇, R₉, and R₁₀ are each —OR₁₄. In some embodiments, R₇, R₉, and R₁₀ are each —OH.

In some embodiments of the compounds of formula (I) or (II), R₁₄ is hydrogen or (C₁-C₆)alkyl.

In some embodiments of the compounds of formula (I) or (II), X is O or N(R₁₄). In some embodiments, X is O.

In some embodiments of the compounds of formula (I) or (II), the compound is

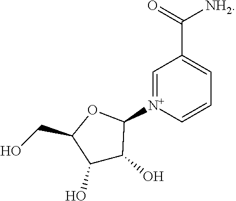 [SMILES: NC(=O)c1ccc[n+]([C@@H]2O[C@H](CO)[C@@H](O)[C@H]2O)c1]

Pterostilbene is a stilbenoid and an analog of polyphenol reservatrol that has better bioavailability due to the presence of two methoxy groups that allow it to have increased lipophilic and oral absorption as well as a longer half-life due to reduced oxidation. The chemical structure of pterostilbene is provided below:

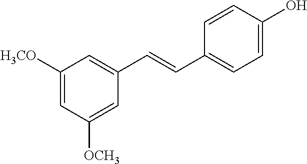 [SMILES: COc1cc(/C=C/c2ccc(O)cc2)cc(OC)c1]

In some embodiments, provided herein are pharmaceutical compositions comprising a compound represented by Formula (III) or a pharmaceutically acceptable salt thereof:

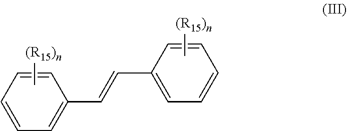

wherein, independently for each occurrence:

R₁₅ is selected from halogen, —CN, —NO₂, —OR₁₆, —N(R₁₆)ₚ, —S(O)₂(OR₁₆), —S(O)OR₁₆, substituted or unsubstituted (C₁-C₆)alkyl, cycloalkyl, heterocycloalkyl, aryl, heteroaryl, aralkyl, and heteroaralkyl;

R₁₆ is selected from hydrogen, (C₁-C₆)alkyl, cycloalkyl, heterocycloalkyl, aryl, heteroaryl, aralkyl, and heteroaralkyl;

n is an integer from 0 to 5; and

p is 2 or 3;

provided that at least one n is 1; and at least one R₁₅ is —OR₁₆;

provided that the compound of formula (III) is not

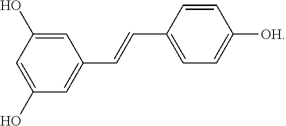 [SMILES: Oc1ccc(/C=C/c2cc(O)cc(O)c2)cc1]

In some embodiments of the compounds of formula (III), R₁₅ is selected from, halogen, —CN, —NO₂, —OR₁₆, —N(R₁₆)ₚ, and substituted or unsubstituted (C₁-C₆)alkyl. In some embodiments, R₁₅ is selected from —OR₁₆, —N(R₁₆)ₚ, and unsubstituted (C₁-C₆)alkyl. In some embodiments, R₁₅ is selected from substituted or unsubstituted (C₁-C₆)alkyl, cycloalkyl, heterocycloalkyl, aryl, heteroaryl, aralkyl, and heteroaralkyl. In some embodiments, R₁₅ is —OR₁₆. In some embodiments, R₁₅ is —OR₁₆; and R₁₆ is hydrogen or (C₁-C₆)alkyl. In some embodiments, R₁₅ is —OR₁₆; and R₁₆ is (C₁-C₆)alkyl, cycloalkyl, heterocycloalkyl, aryl, heteroaryl, aralkyl, and heteroaralkyl. In some embodiments, R₁₅ is —OR₁₆; and R₁₆ is (C₁-C₆)alkyl. In some embodiments, R₁₅ is —OR₁₆; and RIG is (C₁-C₆)alkyl, cycloalkyl, or heterocycloalkyl.

In some embodiments, n is 1, 2, or 3. In some embodiments, n is 1 or 2.

In some embodiments, p is 2. In some embodiments, p is 3.

In one aspect, the provided herein are pharmaceutically acceptable compositions which comprise a therapeutically-effective amount of one or more of the compounds described herein (e.g., nicotinamide riboside and/or pterostilbene), formulated together with one or more pharmaceutically acceptable carriers (additives) and/or diluents. In another aspect, the agents described herein can be administered as such, or administered in mixtures with pharmaceutically acceptable carriers and can also be administered in conjunction with other agents. Conjunctive therapy thus includes sequential, simultaneous and separate, or co-administration of one or more compounds of the invention, wherein the therapeutic effects of the first administered has not entirely disappeared when the subsequent compound is administered.

As described in detail below, the pharmaceutical compositions described herein may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; or (3) sublingually.

In some embodiments, the composition comprises additional agents. For example, the composition may comprise a nutritional agent, such as an antioxidant. Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

The formulations of the compounds described herein may be presented in unit dosage form and may be prepared by any methods well known in the art of pharmacy. The amount of active ingredient which can be combined with a carrier material to produce a single dosage form will vary depending upon the host being treated and the particular mode of administration. The amount of active ingredient which can be combined with a carrier material to produce a single dosage form will generally be that amount of the agent which produces a therapeutic effect.

In certain embodiments, a formulation described herein comprises an excipient, including, but not limited to, cyclodextrins, liposomes, micelle forming agents, e.g., bile acids, and polymeric carriers, e.g., polyesters and polyanhydrides; and an agent of the invention. In some embodiments, an aforementioned formulation renders orally bioavailable an agent of the invention. Methods of preparing these formulations or compositions may include the step of bringing into association a compound of the invention with the carrier and, optionally, one or more accessory ingredients.

Liquid dosage forms for oral administration of the formulations provided herein include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations provided herein suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the invention as an active ingredient. A compound of the invention may also be administered as a bolus, electuary, or paste.

In solid dosage forms of the invention for oral administration (e.g., capsules, tablets, pills, dragees, powders, granules and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol, glycerol monostearate, and non-ionic surfactants; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions described herein, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. Compositions described herein may also be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Pharmaceutical compositions provided herein suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

### Therapeutic Methods

Provided herein are methods and compositions related to preventing, treating, or slowing the progression of glaucoma in a subject by administering to the subject (e.g., orally administering to the subject) a composition comprising a compound of Formula I or Formula II (e.g., nicotinamide riboside), and/or a compound of Formula III (e.g., pterostilbene). The subject may be male or female. In some embodiments, the subject is a mammal, preferably, a human.

Glaucoma is a group of complex, multifactorial diseases characterized by the progressive dysfunction and loss of retinal ganglion cells (RGCs), leading to vision loss. Glaucoma is a complex disease involving multiple insults. However, mechanical axon damage and local inflammation are two important contributors in glaucoma. In some aspects, provided herein are methods and compositions related to reducing progressive dysfunction of retinal ganglion cells and/or reducing or slowing the loss of retinal ganglion cells in a subject in need thereof, by administering to the subject (e.g., orally administering to the subject) a composition comprising a compound of Formula I or Formula II (e.g., nicotinamide riboside), and/or a compound of Formula III (e.g., pterostilbene). In some aspects, provided herein are methods and compositions related treating and/or reducing intraocular eye pressure or inflammation in a subject in need thereof, by administering to the subject (e.g., orally administering to the subject) a composition comprising a compound of Formula I or Formula II (e.g., nicotinamide riboside), and/or a compound of Formula III (e.g., pterostilbene).

The glaucoma may be open angle glaucoma, angle closure glaucoma, or normal-tension glaucoma. Open-angle glaucoma, the most common form of glaucoma, accounting for at least 90% of all glaucoma cases, is caused by the slow clogging of the drainage canals, resulting in increased eye pressure. “Open-angle” means that the angle where the iris meets the cornea is as wide and open. Open-angle glaucoma is also called primary or chronic glaucoma. Angle-closure glaucoma, a less common form of glaucoma, is caused by blocked drainage canals, resulting in a sudden rise in intraocular pressure, and is characterized by a closed or narrow angle between the iris and cornea. It is also called acute glaucoma or narrow-angle glaucoma. Unlike open-angle glaucoma, angle-closure glaucoma is a result of the angle between the iris and cornea closing. Normal-tension glaucoma (NTG), also called low-tension or normal-pressure glaucoma, is characterized by a damaged optic nerve even though the eye pressure is not very high. In certain embodiments, the glaucoma is open angle glaucoma. In certain embodiments, the glaucoma is angle closure glaucoma.

The glaucoma may be secondary glaucoma, pigmentary glaucoma, pseudoexfoliative glaucoma, traumatic glaucoma, neovascular glaucoma, irido corneal endothelial syndrome (ICE), congenital glaucoma, or uveitic glaucoma. Secondary glaucoma refers to any case in which another disease causes or contributes to increased eye pressure, resulting in optic nerve damage and vision loss. Secondary glaucoma can occur as the result of an eye injury, inflammation, tumor, or in advanced cases of cataract or diabetes. It can also be caused by certain drugs, such as steroids. This form of glaucoma may be mild or severe. In other cases, an abnormal drainage system may be the result of some other disease in the eye which results in secondary glaucoma. In these cases, the glaucoma may be associated with recognizable iris (the colored part of the eye), corneal, or other eye problems.

Pigmentary glaucoma is a form of secondary open-angle glaucoma. It occurs when the pigment granules that are in the back of the iris (the colored part of the eye) break into the clear fluid produced inside the eye. These tiny pigment granules flow toward the drainage canals in the eye and slowly clog them. This causes eye pressure to rise.

Pseudoexfoliative glaucoma is a form of secondary open-angle glaucoma occurs when a flaky, dandruff-like material peels off the outer layer of the lens within the eye. The material collects in the angle between the cornea and iris and can clog the drainage system of the eye, causing eye pressure to rise.

Traumatic glaucoma is an injury to the eye may cause secondary open-angle glaucoma. Traumatic glaucoma can occur immediately after the injury or years later. It can be caused by blunt injuries that bruise the eye (called blunt trauma) or by injuries that penetrate the eye. Conditions such as severe nearsightedness, previous injury, infection, or prior surgery may make the eye more vulnerable to a serious eye injury and traumatic glaucoma.

Neovascular glaucoma is abnormal formation of new blood vessels on the iris and over the eye's drainage channels can cause a form of secondary open-angle glaucoma. Neovascular glaucoma is associated with other abnormalities, most often diabetes. The new blood vessels block the eye's fluid from exiting through the trabecular meshwork (the eye's drainage canals), causing an increase in eye pressure.

Irido Corneal Endothelial Syndrome (ICE) is a rare form of glaucoma usually appears in only one eye, rather than both. Cells on the back surface of the cornea spread over the eye's drainage tissue and across the surface of the iris, increasing eye pressure and damaging the optic nerve. These corneal cells also form adhesions that bind the iris to the cornea, further blocking the drainage channels. Symptoms can include hazy vision upon awakening and the appearance of halos around lights.

Congenital glaucoma or childhood glaucoma refers to the presence of glaucoma in a child, and occurs in 1 out of every 10,000 births in the United States. Congenital glaucoma is the common term used for a glaucoma diagnosed in infancy or early childhood. This glaucoma is caused by abnormal intraocular fluid drainage from the eye as a result of a blocked or defective trabecular meshwork (the mesh-like drainage canals in the eye). Congenital glaucoma may be due to a hereditary defect or abnormal development during pregnancy.

Provided herein are methods and compositions related to treating, preventing, and/or improving a symptom of glaucoma by administering (e.g., orally administering to the subject) a composition comprising a compound of Formula I or Formula II (e.g., nicotinamide riboside), and/or a compound of Formula III (e.g., pterostilbene). Symptoms of may include hazy or blurred vision, appearance of rainbow-colored circles around bright lights, severe eye and head pain, nausea or vomiting, or sudden sight loss. A symptom may be any symptom disclosed herein.

Clinically, glaucoma can be assessed and monitored using any of a number of structural (anatomical) and functional tests to measure eye pressure or other measures of visual field, including, without limitation: tonometry, ophthalmoscopy, perimetry, gonioscopy, or pachymetry. Tonometry measures the pressure within your eye. During tonometry, eye drops are used to numb the eye, and a device called a tonometer to measure the inner pressure of the eye. The range for normal pressure is 12-22 mm Hg. Most glaucoma cases are diagnosed with pressure exceeding 20 mm Hg. However, some subjects may have glaucoma at pressures between 12-22 mm Hg. In some embodiments, a subject may have an eye pressure of equal to or over 20 mm Hg. In some embodiments, a subject may have an eye pressure of lower than 20 mm Hg. Ophthalmoscopy is a diagnostic procedure to measure the damage to optic nerve as a result of glaucoma. The pupil is dilated in order to examine the shape and color of the optic nerve. An additional test used to evaluate the existence of progression of glaucoma is perimetry. Perimetry is a visual field test that produces a map of your complete field of vision. Gonioscopy is a diagnostic exam helps determine whether the angle where the iris meets the cornea is open and wide or narrow and closed. During the exam, a hand-held contact lens is gently placed on the eye. This contact lens contains a mirror that shows if the angle between the iris and cornea is closed and blocked (a possible sign of angle-closure or acute glaucoma) or wide and open (a possible sign of open-angle, chronic glaucoma).

The various methods disclosed herein can be methods for improving vision or slowing vision loss in a subject with glaucoma, and/or methods for improving eye pressure or the visual field in a subject, such as a subject with glaucoma. Thus, in certain embodiments, the methods of treatment disclosed herein include methods for stabilizing eye pressure or the visual field of a subject, whereby the patient's eye pressure or visual field (as measured by a test disclosed herein) after a therapy (e.g., after about one month, about three months, after about six months, after about nine months, after about one year, after about two years, or after about three years) comprising administration of a composition disclosed herein is at least about 10%, at least about 25%, at least about 40%, at least about 50%, or even at least about 60% improved relative to a control patient not receiving a therapy disclosed herein. Alternatively, the patient's eye pressure or visual field (as measured by a test disclosed herein) after a therapy (e.g., after about one month, about three months, after about six months, after about nine months, after about one year, after about two years, or after about three years) comprising administration of a composition disclosed herein is at least about 2%, at least about 5%, at least about 10%, at least about 25%, at least about 40%, at least about 50%, or even at least about 60% improved than the earlier assessment.

Provided herein are methods and compositions related to treating glaucoma in a subject by administering to the subject (e.g., orally administering to the subject) a composition comprising a compound of Formula I or Formula II (e.g., nicotinamide riboside), and/or a compound of Formula III (e.g., pterostilbene) conjointly with a second therapeutic for glaucoma. In some embodiments, the second therapeutic is a beta-blocker. In some embodiments, the second therapeutic is a prostaglandin analog. In some embodiments, the second therapeutic is an alpha-adrenergic agonist. In some embodiments, the second therapeutic is a carbonic anhydrase inhibitor. In some embodiments, the second therapeutic is a parasympathomimetic. In some embodiments, the second therapeutic is epinephrine. In some embodiments, the second therapeutic is a hyperosmotic agent.

The second therapeutic may be Xalatan® (latanoprost), Lumigan® (bimatoprost), Travatan Z® (Travoprost), and Zioptan™ (tafluprost), timolol, Alphagan®P (brimonidine), Iopidine, Trusopt® (dorzolamide), Azopt® (brinzolamide), Diamox (acetazolamide), and Neptazane® (methazolamide), Cosopt®, dorzolamide, Cosopt®, Combigan®, or Simbrinza®.

Actual dosage levels and administration regimen of the compositions disclosed herein may be varied so as to obtain an amount of a compound of Formula I or Formula II (e.g., nicotinamide riboside) and/or a compound of Formula III (e.g., pterostilbene) that is effective to achieve the desired therapeutic response for a particular patient, composition, and mode of administration, without being toxic to the patient. In some embodiments, the subject continuously self-administers the compounds disclosed herein. In other embodiments, the subject may take a compound disclosed herein as needed.

In some embodiments, administration of the composition comprises administration of the composition in one or more dose(s). In some embodiments, administration of the composition comprises administration of the composition in one or more, five or more, ten or more, twenty or more, thirty or more, forty or more, fifty or more, one hundred or more, or one thousand or more dose(s). In some embodiments, the dose comprises at least 25 mg, at least 50 mg, at least 75 mg, at least 100 mg, at least 125 mg, at least 150 mg, at least 200 mg, at least 225 mg, at least 250 mg, at least 275 mg, at least 300 mg, at least 325 mg, at least 350 mg, at least 375 mg, at least 400 mg, at least 425 mg, at least 450 mg, at least 475 mg, at least 500 mg, at least 550 mg, at least 600 mg, at least 650 mg, at least 700 mg, at least 750 mg, at least 800 mg, or at least 850 mg of a compound of Formula I or Formula II (e.g., nicotinamide riboside). In some embodiments, the dose comprises at least 5 mg, at least 10 mg, at least 15 mg, at least 20 mg, at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 90 mg, at least 95 mg, at least 100 mg, at least 110 mg, at least 120 mg, at least 130 mg, at least 140 mg, at least 150 mg, at least 160 mg, least 170 mg, at least 180 mg, at least 190 mg, at least 200 mg, or at least 250 mg of a compound of formula III (e.g., pterostilbene).

The compositions disclosed herein may be administered over any period of time effective to achieve the desired therapeutic response for a particular patient, composition, and mode of administration, without being toxic to the patient. The period of time may be at least 1 day, at least 10 days, at least 20 days, at least 30, days, at least 60 days, at least three months, at least six months, at least a year, at least three years, at least five years, or at least ten years. The dose may be administered when needed, sporadically, or at regular intervals. For example, the dose may be administered monthly, weekly, biweekly, triweekly, once a day, or twice a day.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A method of preventing or treating glaucoma in a subject comprising administering to the subject a composition comprising nicotinamide riboside.

2. The method of claim 1, wherein the composition further comprises pterostilbene.

3. The method of claim 1 or 2, wherein the glaucoma is open angle glaucoma, angle closure glaucoma, normal tension glaucoma, secondary glaucoma, pigmentary glaucoma, pseudoexfoliative glaucoma, traumatic glaucoma, neovascular glaucoma, irido corneal endothelial syndrome (ICE) or uveitic glaucoma.

4. The method of claim 3, wherein the glaucoma is open angle glaucoma.

5. The method of claim 3, wherein the glaucoma is angle closure glaucoma.

6. A method of slowing the progression of glaucoma in a subject comprising administering to the subject a composition comprising nicotinamide riboside.

7. The method of claim 6, wherein the composition further comprises pterostilbene.

8. The method of claim 6 or 7, wherein the glaucoma is open angle glaucoma, angle closure glaucoma, normal tension glaucoma, secondary glaucoma, pigmentary glaucoma, pseudoexfoliative glaucoma, traumatic glaucoma, neovascular glaucoma, irido corneal endothelial syndrome (ICE) or uveitic glaucoma.

9. The method of claim 8, wherein the glaucoma is open angle glaucoma.

10. The method of claim 8, wherein the glaucoma is angle closure glaucoma.

11. A method of treating or preventing a symptom of glaucoma in a subject comprising administering to the subject a composition comprising nicotinamide riboside.

12. The method of claim 11, wherein the composition further comprises pterostilbene.

13. The method of claim 11 or 12, wherein the symptom is hazy or blurred vision, appearance of rainbow-colored circles around bright lights, severe eye and head pain, nausea or vomiting, or sudden sight loss.

14. A method of reducing intraocular pressure or inflammation in a subject in need thereof, comprising administering to the subject a composition comprising nicotinamide riboside.

15. The method of claim 14, wherein the composition further comprises pterostilbene.

16. A method of slowing or treating progressive intraocular ganglion cell degradation or loss in a subject in need thereof, comprising administering to the subject a composition comprising nicotinamide riboside.

17. The method of claim 16, wherein the composition further comprises pterostilbene.

18. The method of any one of the preceding claims, wherein the method comprises conjointly administering a second therapeutic to the subject.

19. The method of claim 18, wherein the second therapeutic is a beta-blocker, alpha-adrenergic agonists, carbonic anhydrase inhibitors, parasympathomimetics, epinephrine, or a hyperosmotic agent.

20. The method of any one of claims 1 to 19, wherein the administration of the composition comprises administering one or more doses of the composition.

21. The method of claim 20, wherein each dose of the composition comprises at least 200 mg of nicotinamide riboside.

22. The method of claim 20, wherein each dose of the composition comprises at least 250 mg of nicotinamide riboside.

23. The method of claim 20, wherein each dose of the composition comprises at least 300 mg of nicotinamide riboside.

24. The method of claim 20, wherein each dose of the composition comprises at least 350 mg of nicotinamide riboside.

25. The method of claim 20, wherein each dose of the composition comprises at least 400 mg of nicotinamide riboside.

26. The method of claim 20, wherein each dose of the composition comprises at least 450 mg of nicotinamide riboside.

27. The method of claim 20, wherein each dose of the composition comprises at least 500 mg of nicotinamide riboside.

28. The method of claim 20, wherein each dose of the composition comprises at least 550 mg of nicotinamide riboside.

29. The method of any one of claims 20 to 28, wherein each dose of the composition comprises at least 15 mg of pterostilbene.

30. The method of any one of claims 20 to 28, wherein each dose of the composition comprises at least 25 mg of pterostilbene.

31. The method of any one of claims 20 to 28, wherein each dose of the composition comprises at least 50 mg of pterostilbene.

32. The method of any one of claims 20 to 28, wherein each dose of the composition comprises at least 75 mg of pterostilbene.

33. The method of any one of claims 20 to 28, wherein each dose of the composition comprises at least 100 mg of pterostilbene.

34. The method of any one of claims 20 to 28, wherein each dose of the composition comprises at least 125 mg of pterostilbene.

35. The method of any one of claims 20 to 28, wherein each dose of the composition comprises at least 150 mg of pterostilbene.

36. The method of any one of claims 20 to 35, wherein two or more doses of the composition are administered.

37. The method of any one of claims 20 to 36, wherein thirty or more doses of the composition are administered.

38. The method of any one of claims 20 to 37, wherein fifty or more doses of the composition are administered.

39. The method of any one of claims 20 to 38, wherein one hundred or more doses of the composition are administered.

40. The method of any one of claims 20 to 39, wherein the dose of the composition is administered at least once a week.

41. The method of any one of claims 20 to 39, wherein the dose is administered at least twice a week.

42. The method of any one of claims 20 to 39, wherein the dose is administered at least three times a week.

43. The method of any one of claims 20 to 39, wherein the dose is administered at least once a day.

44. The method of any one of claims 20 to 39, wherein the dose is administered at least twice a day.

45. The method of any one of claims 40 to 44, wherein the doses are administered for at least 7 days.

46. The method of any one of claims 40 to 44, wherein the doses are administered for at least 30 days.

47. The method of any one of claims 40 to 44, wherein the doses are administered for at least 60 days.

48. The method of any one of claims 40 to 44, wherein the doses are administered for at least 90 days.

49. The method of any one of claims 1 to 48, wherein the composition is formulated as a pill, a tablet, or a capsule.

50. The method of any one of claims 1 to 49, wherein the composition is administered orally.

51. The method of any one of claims 1 to 50, wherein the composition is self-administered.
